(19) United States
(12) Patent Application Publication
(10) Pub. No.: US 2024/0366330 A1
(43) Pub. Date: Nov. 7, 2024

(54) Connection Systems and Methods for Establishing Optical and Electrical Connections Through A Drape

(71) Applicant: Bard Access Systems, Inc., Salt Lake City, UT (US)

(72) Inventors: Chase Thompson, Bountiful, UT (US); William Robert McLaughlin, Bountiful, UT (US)

(21) Appl. No.: 18/773,870

(22) Filed: Jul. 16, 2024

Related U.S. Application Data
(63) Continuation of application No. 18/235,334, filed on Aug. 17, 2023, now Pat. No. 12,059,228.
Application No. 18/235,334 is a continuation of application No. 16/923,912, filed on Jul. 8, 2020, now Pat. No. 11,737,848.
(60) Provisional application No. 62/879,966, filed on Jul. 29, 2019.

(51) Int. Cl. A61B 46/23; A61B 5/00

(52) U.S. Cl. CPC A61B 46/23; A61B 5/0084; A61B 5/6852; A61B 2046/234

(57) ABSTRACT

Connection systems and methods for establishing optical and electrical connections through a drape are disclosed. A connection system can include a plug and a receptacle, the plug being configured to insert into the receptacle with the drape therebetween. The plug can include an optical terminal extending from a plug housing and an electrical terminal extending from the plug housing. The electrical terminal can be configured as a piercing element for piercing the drape. The receptacle can include an optical receiver within a receptacle housing and an electrical receiver within the receptacle housing. The optical receiver can be configured to form the optical connection with the optical terminal, and the electrical receiver can be configured to form the electrical connection with the electrical terminal when the plug is inserted into the receptacle with the drape therebetween.

### PRIORITY

This application is a continuation of U.S. patent application Ser. No. 18/235,334, filed Aug. 17, 2023, which is a continuation of U.S. patent application Ser. No. 16/923,912, filed Jul. 8, 2020, now U.S. Pat. No. 11,737,848, which claims the benefit of priority to U.S. Provisional Application No. 62/879,966, filed Jul. 29, 2019, each of which is incorporated by reference in its entirety into this application.

### BACKGROUND

At times, a tip of a peripherally inserted central catheter (“PICC”) or central venous catheter (“CVC”) can move, becoming displaced from an ideal position in a patient's superior vena cava (“SVC”). A clinician believing such a PICC or CVC has displaced typically checks for displacement by chest X-ray and replaces the PICC or CVC if necessary. Because X-rays expose patients to ionizing radiation, medical devices such as PICCs and CVCs are being developed with integrated optical-fiber stylets for clinicians to easily and safely check for displacement thereof by way of shape sensing with the optical-fiber stylets. However, in order for the clinicians to check for displacement, the PICCs or CVCs, which are sterile as provided, need to be optically and electrically connected to non-sterile capital equipment without compromising sterile conditions. Therefore, there is a need for a connection system that allows for single-use medical devices such as the foregoing PICCs and CVCs to be optically and electrically connected to non-sterile capital equipment without compromising sterile conditions.

Disclosed herein are connection systems and methods thereof that address at least the foregoing need.

### SUMMARY

Disclosed herein is a connection system for establishing both an optical connection and an electrical connection through a drape. The connection system includes, in some embodiments, a plug and a receptacle, wherein the plug is configured to insert into the receptacle with the drape therebetween. The plug includes a plug housing, an optical terminal extending from the plug housing, and an electrical terminal extending from the plug housing. The electrical terminal is configured as a piercing element for piercing the drape. The receptacle includes a receptacle housing, an optical receiver within the receptacle housing, and an electrical receiver within the receptacle housing. The optical receiver is configured to form the optical connection with the optical terminal when the plug is inserted into the receptacle with the drape therebetween. The electrical receiver is configured to form the electrical connection with the electrical terminal when the plug is inserted into the receptacle with the drape therebetween.

In some embodiments, the piercing element includes a longitudinal cutout between the plug housing and a tip of the piercing element. The optical terminal extends from the plug housing into the longitudinal cutout of the piercing element.

In some embodiments, the piercing element is configured to deflect away from a longitudinal axis of the plug to expose the optical terminal.

In some embodiments, the electrical receiver includes a channel configured to deflect the piercing element away from the longitudinal axis of the plug upon inserting the plug into the receptacle. Deflecting the piercing element away from the longitudinal axis of the plug exposes the optical terminal for insertion into the optical receiver and formation of the optical connection.

In some embodiments, the channel of the electrical receiver is configured to keep the piercing element deflected away from the longitudinal axis of the plug. The channel of the electrical receiver is also configured form the electrical connection with an electrical contact within the electrical receiver upon fully inserting the plug into the receptacle.

In some embodiments, the piercing element is configured to deflect toward a longitudinal side of the plug.

In some embodiments, the electrical receiver includes a channel configured to deflect the piercing element toward the longitudinal side of the plug upon inserting the plug into the receptacle. Deflect the piercing element toward the longitudinal side of the plug exposes the optical terminal for insertion into the optical receiver and formation of the optical connection.

In some embodiments, the channel of the electrical receiver is configured to keep the piercing element deflected toward the longitudinal side of the plug. The channel of the electrical receiver is also configured to form the electrical connection with an electrical contact within the electrical receiver upon fully inserting the plug into the receptacle.

In some embodiments, the optical terminal includes an optical fiber extending from the plug housing into a ferrule, and the optical receiver includes an optical fiber disposed in a ferrule at a closed end of the optical receiver.

In some embodiments, the ferrules connect in a straight sleeve-type butt-joint connection.

In some embodiments, the ferrules connect in a tapered sleeve-type butt-joint connection.

In some embodiments, the plug is configured to simultaneously make both the optical and electrical connections with the receptacle from a sterile field established by the drape without compromising sterility of the sterile field.

Also disclosed herein is a connection system for establishing both an optical connection and an electrical connection through a drape. The connection system includes, in some embodiments, a plug and a receptacle, wherein the plug is configured to insert into the receptacle with the drape therebetween. The plug includes a plug housing, an optical terminal extending from the plug housing, and an electrical terminal extending from the plug housing. The optical terminal includes an optical fiber extending from the plug housing into a ferrule. The electrical terminal is configured as a piercing element for piercing the drape. The piercing element includes a longitudinal cutout between the plug housing and a tip of the piercing element, and the optical terminal extends from the plug housing into the longitudinal cutout of the piercing element. The receptacle includes a receptacle housing, an optical receiver within the receptacle housing, and an electrical receiver within the receptacle housing. The optical receiver is configured to form the optical connection with the optical terminal when the plug is inserted into the receptacle with the drape therebetween. The optical receiver including an optical fiber disposed in a ferrule at a closed end of the optical receiver. The electrical receiver is configured to form the electrical connection with the electrical terminal when the plug is inserted into the receptacle with the drape therebetween.

In some embodiments, the electrical receiver includes a channel configured to deflect the piercing element away from a longitudinal axis of the plug or toward a longitudinal side of the plug upon inserting the plug into the receptacle. Deflecting the piercing element away from the longitudinal axis of the plug or toward the longitudinal side of the plug exposes the optical terminal for insertion into the optical receiver and formation of the optical connection.

In some embodiments, the channel of the electrical receiver is configured to keep the piercing element deflected away from the longitudinal axis or toward the longitudinal side of the plug. The channel of the electrical receiver is also configured form the electrical connection with an electrical contact within the electrical receiver upon fully inserting the plug into the receptacle.

In some embodiments, the plug is configured to simultaneously make both the optical and electrical connections with the receptacle from a sterile field established by the drape without compromising sterility of the sterile field.

Also disclosed herein is a method for establishing both an optical connection and an electrical connection through a drape including, in some embodiments, establishing a sterile field with the drape; piercing the drape from a sterile side thereof with an electrical terminal configured as a piercing element that extends from a plug, wherein the piercing element includes a longitudinal cutout between a housing of the plug and a tip of the piercing element in which cutout an optical terminal that extends from the plug is disposed; inserting the plug into a receptacle on a non-sterile side of the drape, wherein the receptacle includes an optical receiver and an electrical receiver within a receptacle housing configured to form the optical connection with the optical terminal and the electrical connection with the piercing element, respectively; and establishing both the optical and electrical connections through the drape without compromising sterility of the sterile field.

In some embodiments, piercing the drape with the piercing element simultaneously advances the optical terminal through the drape in accordance with disposition of the optical terminal in the cutout of the piercing element.

In some embodiments, inserting the plug into the receptacle includes inserting the piercing element into a channel of the electrical receiver. The channel is configured to deflect the piercing element away from a longitudinal axis of the plug or toward a longitudinal side of the plug. Deflecting the piercing element away from the longitudinal axis of the plug or toward the longitudinal side of the plug exposes the optical terminal.

In some embodiments, inserting the plug into the receptacle includes forming a butt-joint connection between a ferrule-covered portion of the optical terminal and a ferrule-covered portion of optical fiber at a closed end of the optical receiver.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a CVC including an integrated optical-fiber stylet in accordance with some embodiments.

FIG. 2 illustrates a connection system including a plug and a receptacle in accordance with some embodiments.

FIG. 3 illustrates deflection of a piercing element of the plug in accordance with some embodiments.

FIG. 4 illustrates deflection of a piercing element of the plug in accordance with some embodiments.

FIG. 5A illustrates insertion of the plug through a drape and into the receptacle in accordance with some embodiments.

FIG. 5B illustrates continued insertion of the plug through the drape and into the receptacle in accordance with some embodiments.

FIG. 5C illustrates complete insertion of the plug through the drape and into the receptacle in accordance with some embodiments.

FIG. 6 illustrates a cross-section of the plug completely inserted into the receptacle forming both optical electrical connections in accordance with some embodiments.

FIG. 7A illustrates insertion of the plug through a drape and into the receptacle in accordance with some embodiments.

FIG. 7B illustrates complete insertion of the plug through the drape and into the receptacle in accordance with some embodiments.

FIG. 8A illustrates a straight sleeve-type butt-joint connection of an optical connection in accordance with some embodiments.

FIG. 8B illustrates a tapered sleeve-type butt-joint connection of an optical connection in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

Unless defined otherwise, all technical and scientific terms used herein have the same meaning as commonly understood by those of ordinary skill in the art.

As set forth above, there is a need for a connection system that allows for single-use medical devices such as PICCs and CVCs to be optically and electrically connected to non-sterile capital equipment without compromising sterile conditions. Disclosed herein are connection systems and methods thereof that address at least the foregoing need.

For example, a connection system for establishing both an optical connection and an electrical connection through a drape is disclosed herein. The connection system includes, in some embodiments, a plug and a receptacle, wherein the plug is configured to insert into the receptacle with the drape therebetween. The plug includes a plug housing, an optical terminal extending from the plug housing, and an electrical terminal extending from the plug housing. The electrical terminal is configured as a piercing element for piercing the drape. The receptacle includes a receptacle housing, an optical receiver within the receptacle housing, and an electrical receiver within the receptacle housing. The optical receiver is configured to form the optical connection with the optical terminal when the plug is inserted into the receptacle with the drape therebetween. The electrical receiver is configured to form the electrical connection with the electrical terminal when the plug is inserted into the receptacle with the drape therebetween.

These and other features of the connection system provided herein will become more apparent with reference to the accompanying drawings and the following description, which provide particular embodiments of the connection system in greater detail. For context, a medical device including a portion of a connection system is described first followed by connection systems and methods thereof.

### Medical Devices

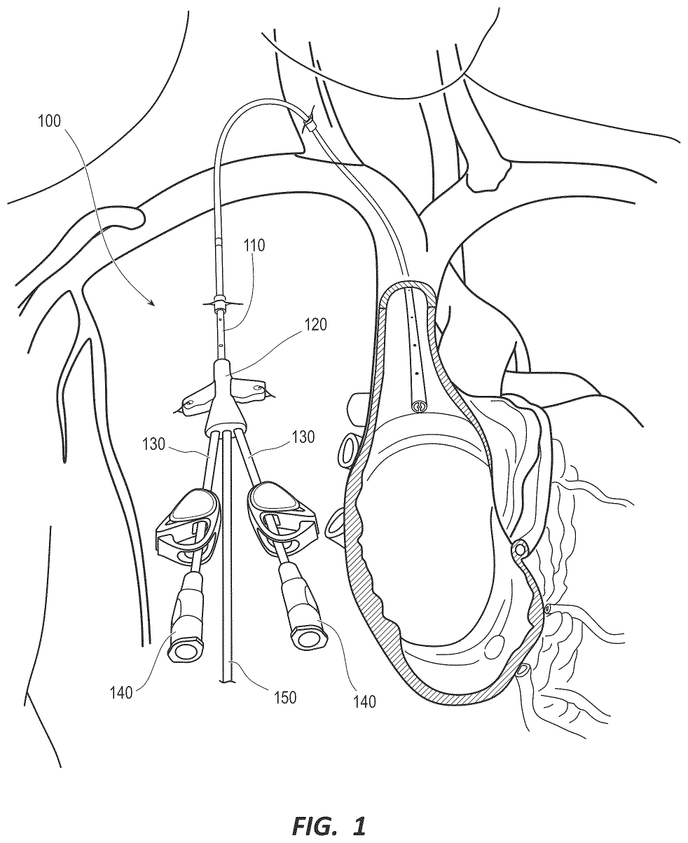

FIG. 1 illustrates a CVC 100 including an integrated optical-fiber stylet in accordance with some embodiments.

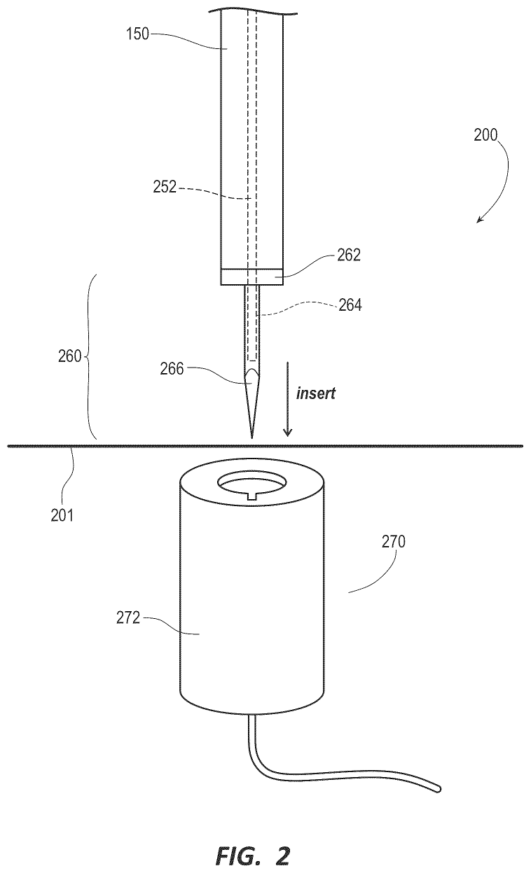

As shown, the CVC 100 includes a catheter tube 110, a bifurcated hub 120, two extension legs 130, and two Luer connectors 140 operably connected in the foregoing order. While not shown in FIG. 1, the catheter tube 110 includes two catheter-tube lumens and the optical-fiber stylet disposed in a longitudinal bead of the catheter tube 110 such as between the two catheter-tube lumens, as extruded. (See, e.g., optical-fiber stylet 252 in FIG. 2.) In addition, in a same or different longitudinal bead of the catheter tube 110, the CVC 100 can further include an electrocardiogram (“ECG”) stylet. The bifurcated hub 120 has two hub lumens correspondingly fluidly connected to the two catheter-tube lumens. Each extension leg of the two extension legs 130 has an extension-leg lumen fluidly connected to a hub lumen of the two hub lumens. The CVC 100 further includes a stylet extension tube 150 extending from the bifurcated hub 120. The stylet extension tube 150 can be a skived portion of the catheter tube 110 including the optical-fiber stylet or the skived portion of the catheter tube 110 disposed in another tube, either of which can terminate in a portion of a connection system such as a connection-system plug as shown in FIG. 2.

While the CVC 100 is provided as a medical device including a portion of a connection system, it should be understood that any of a number of medical devices including catheters such as a PICC can include at least an optical-fiber stylet and a stylet extension tube that terminates in a portion of a connection system for interfacing the optical-fiber stylet with procedural equipment or instrumentation.

### Connection Systems

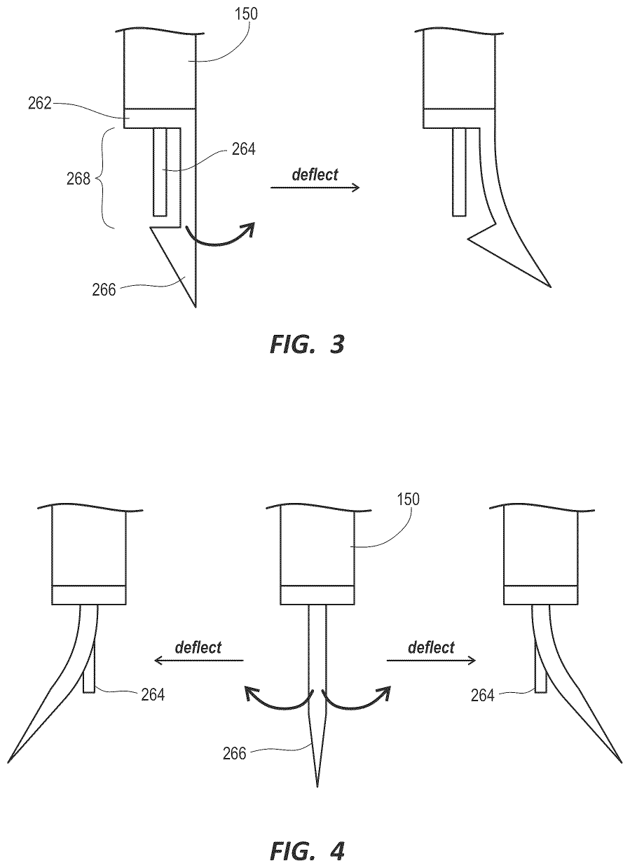

FIG. 2 illustrates a connection system 200 including a plug 260 and a receptacle 270 in accordance with some embodiments. FIGS. 3 and 4 illustrate deflection of a piercing element 266 of the plug 260 in accordance with some embodiments.

As shown in FIG. 2, the connection system 200, which is configured for establishing either an optical connection through a drape 210 or both optical and electrical connections through the drape 201, includes the plug 260 and the receptacle 270. The plug 260 can be at, for example, a proximal-end portion of the stylet extension tube 150 of the CVC 100; however, the plug 260 is not limited to the foregoing medical device as set forth above. From a sterile field established by the drape 201, the plug 260 is configured to insert into the receptacle 270 with the drape 201 therebetween without compromising sterility of the sterile field.

The plug 260 includes a plug housing 262, an optical terminal 264 extending from the plug housing 262, and a piercing element 266 extending from the plug housing 262 for piercing the drape 201. The plug housing 262 can be coupled to, for example, a proximal-end portion of the stylet extension tube 150 of the CVC 100. The piercing element 266 can also be an electrical terminal when the ECG stylet is present, so the electrical terminal is also referenced herein by reference numeral 266. The optical terminal 264 extends from the plug housing 262 into a longitudinal cutout 268 of the piercing element 266 between the plug housing 262 and a tip of the piercing element 266. (See FIG. 3 for the longitudinal cutout 268.) The longitudinal cutout 268 protects the optical terminal 264 from inadvertent damage both before and while piercing the drape 201 with the piercing element 266 to establish the optical connection.

The piercing element 266 is configured to deflect away from a longitudinal axis of the plug 260 to expose the optical terminal 264 as shown in FIG. 3. The piercing element 266 is additionally or alternatively configured to deflect toward one or both longitudinal sides of the plug 260 as shown in FIG. 4.

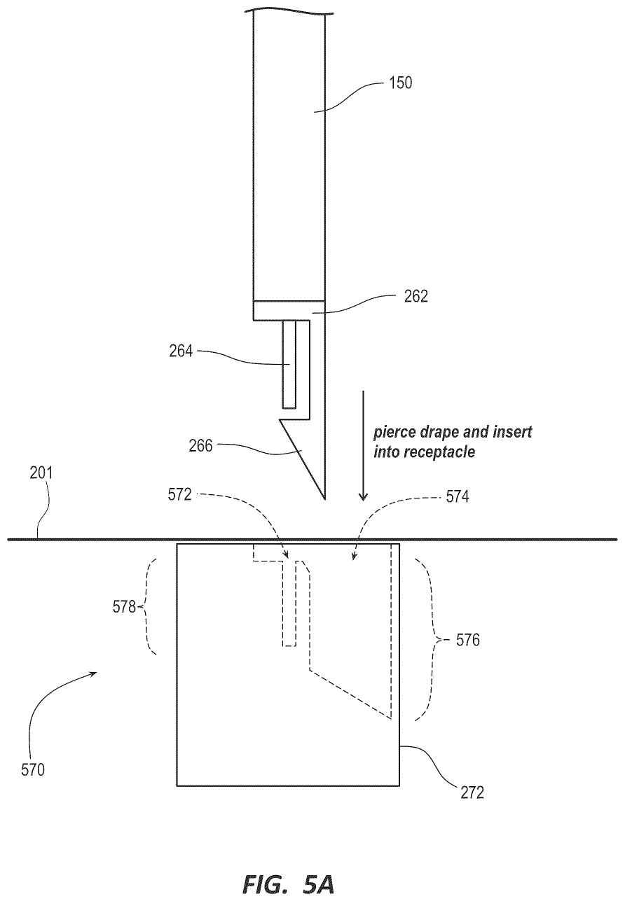

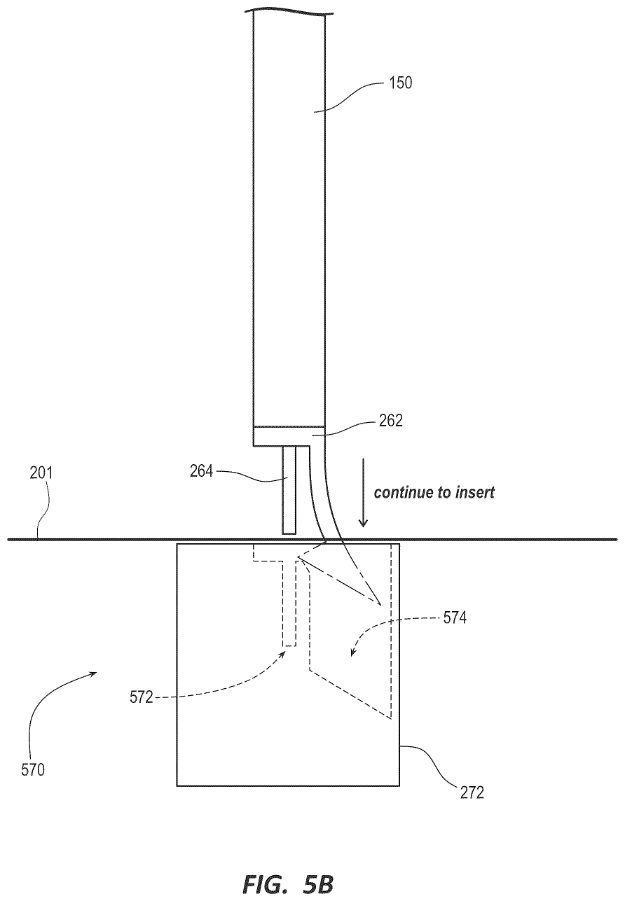

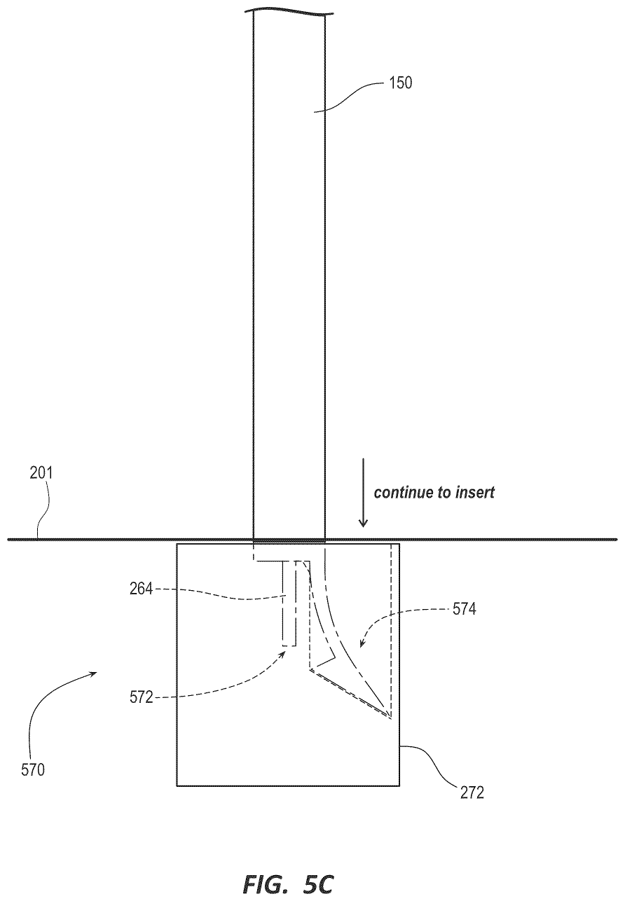

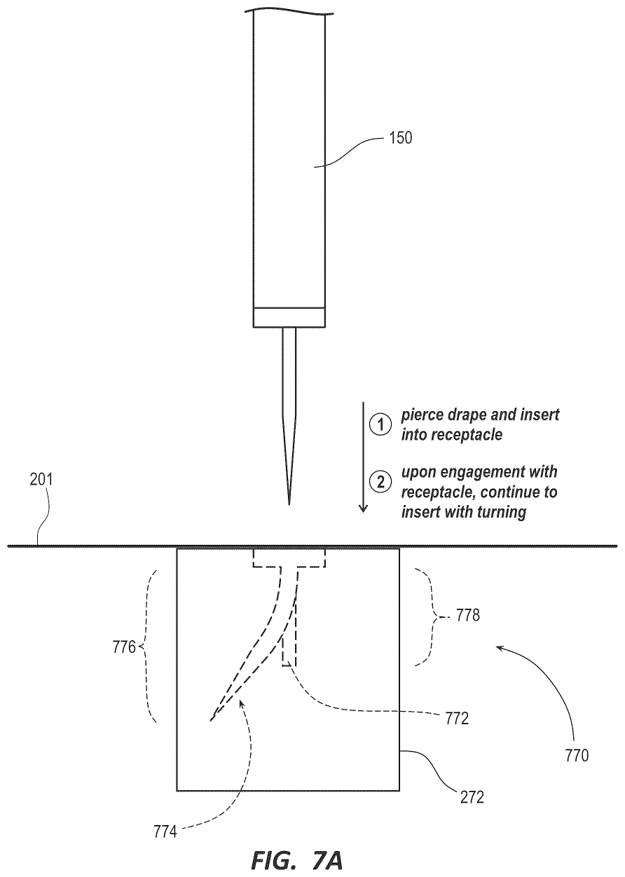

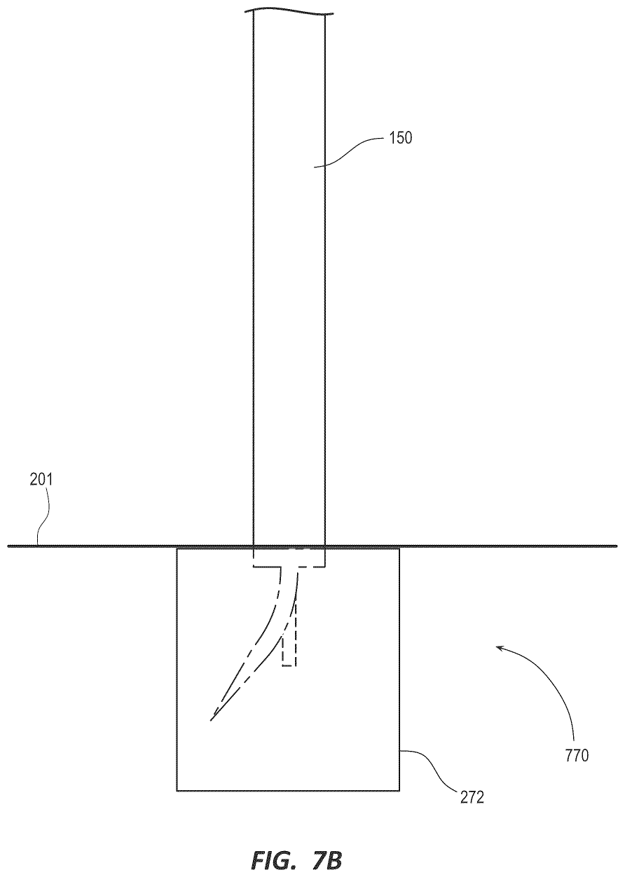

The receptacle 270, which is generic to other receptacles disclosed herein, includes a receptacle housing 272. Depending upon a particular configuration of the receptacle 270 such as that exemplified by receptacles 570 and 770 respectively of FIGS. 5A-5C and FIGS. 7A and 7B, the receptacle 270 also includes an optical receiver 572 or 772 within the receptacle housing 272 and an electrical receiver 574 or 774 within the receptacle housing 272. (See FIGS. 5A-5C for the optical receiver 572 and electrical receiver 574, and see FIGS. 7A and 7B for optical receiver 772 and electrical receiver 774.) The optical receiver 572 or 772 is configured to form the optical connection with the optical terminal 264 when the plug 260 is inserted into the receptacle 570 or 770 with the drape 201 therebetween. The electrical receiver 574 or 774 is configured to form the electrical connection with the electrical terminal 266 when the plug 260 is inserted into the receptacle 570 or 770 with the drape 201 therebetween.

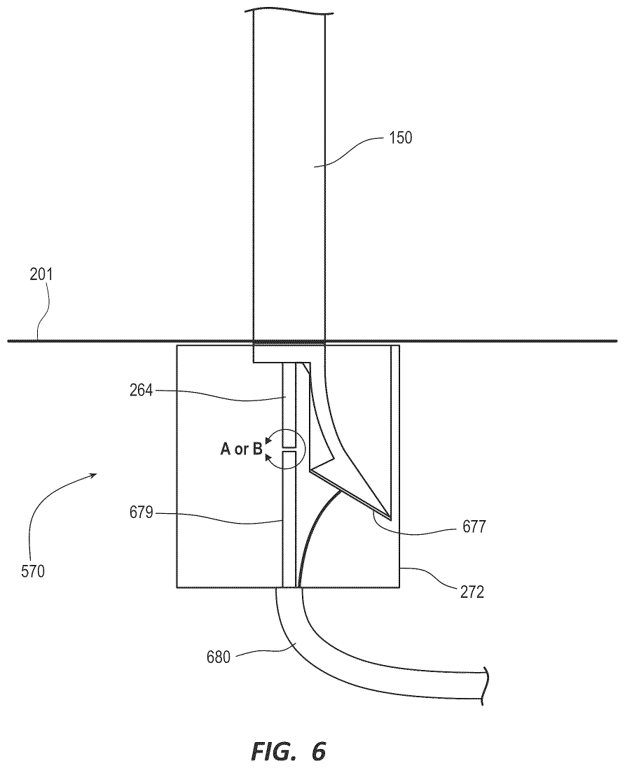

FIGS. 5A-5C illustrate insertion of the plug 260 through the drape 201 and into the receptacle 570 in accordance with some embodiments. FIG. 6 illustrates a cross-section of the plug 260 completely inserted into the receptacle 570 forming both optical electrical connections in accordance with some embodiments.

As shown, the receptacle 570 includes the optical receiver 572 and the electrical receiver 574 within the receptacle housing 272. The electrical receiver 574 includes an electrical-receiver channel 576 configured to deflect the piercing element 266 away from the longitudinal axis of the plug 260 upon inserting the plug 260 into the receptacle 570. Deflecting the piercing element 266 away from the longitudinal axis of the plug 260 exposes the optical terminal 264 for insertion into an optical-receiver channel 578 of the optical receiver 572 and formation of the optical connection. The electrical-receiver channel 576 is further configured to keep the piercing element 266 deflected away from the longitudinal axis of the plug 260 to maintain the optical connection. The electrical-receiver channel 576 is even further configured to form the electrical connection with an electrical contact 677 within the electrical receiver 574 such as at a bottom of the electrical receiver 574 upon fully inserting the plug 260 into the receptacle 570. Likewise, the optical-receiver channel 578 is configured to form the optical connection with an end portion of an optical fiber 679 within the optical receiver 572 such as at a bottom of the optical receiver 572 upon fully inserting the plug 260 into the receptacle 570. The electrical contact 677 is electrically connected (e.g., soldered) to a wire, and both the optical fiber 679 and the wire extend from the receptacle 570 in a same or different cable 680 as shown.

FIGS. 7A and 7B illustrates insertion of the plug 260 through the drape 201 and into a receptacle 770 in accordance with some embodiments. Note, the plug 260 of FIGS. 7A and 7B is that of FIGS. 5A-5C but rotated clockwise around the longitudinal axis of the stylet extension tube 150 about 90°.

As shown, the receptacle 770 includes the optical receiver 772 and the electrical receiver 774 within the receptacle housing 272. The electrical receiver 774 includes an electrical-receiver channel 776 configured to deflect the piercing element 266 toward the longitudinal side of the plug 260 upon inserting the plug 260 into the receptacle 770 and turning the plug 260 upon engagement with the receptacle 770, for example, about a ¼ turn. Deflecting the piercing element 266 toward the longitudinal side of the plug 260 exposes the optical terminal 264 for insertion into an optical-receiver channel 778 of the optical receiver 772 and formation of the optical connection. The electrical-receiver channel 776 is further configured to keep the piercing element 266 deflected toward the longitudinal side of the plug 260 to maintain the optical connection. The electrical-receiver channel 776 is even further configured to form the electrical connection with an electrical contact within the electrical receiver 774 such as at a bottom of the electrical receiver 774 upon fully inserting the plug 260 into the receptacle 770. (For an example electrical contact, see the electrical contact 677 of FIG. 6.) Likewise, the optical-receiver channel 778 is configured to form the optical connection with an end portion of an optical fiber within the optical receiver 772 such as at a bottom of the optical receiver 772 upon fully inserting the plug 260 into the receptacle 770. (For an example optical connection, see the optical connection between the optical terminal 264 and the end portion of the optical fiber 679 in FIG. 6.)

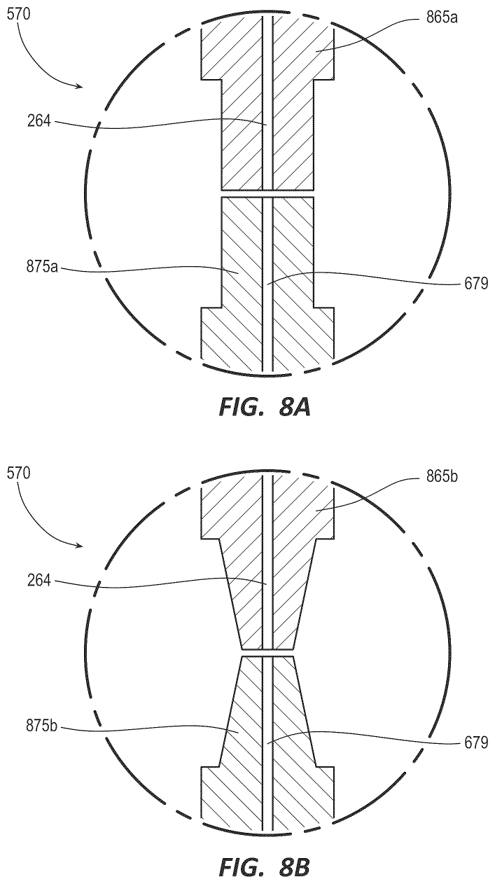

FIG. 8A illustrates a straight sleeve-type butt-joint connection of an optical connection in accordance with some embodiments. FIG. 8B illustrates a tapered sleeve-type butt-joint connection of an optical connection in accordance with some embodiments.

As shown among FIGS. 5A, 7, 8A and 8B, the optical terminal 264, which is a proximal-end portion of an optical fiber such as that of the optical-fiber stylet 252, extends from the plug housing 262 into a ferrule 865a or 865b. The optical receiver 572 includes the optical fiber 679 disposed in a ferrule 875a or 875b at a closed end or bottom of the optical receiver 572. Likewise, the optical receiver 772 can include the optical fiber 679 disposed in the ferrule 875a or 875b at a closed end or bottom of the optical receiver 772. The ferrules 865a and 875a connect in a straight sleeve-type butt-joint connection for the optical connection. The ferrules 865b and 875b connect in a tapered sleeve-type butt-joint connection for the optical connection.

### Methods

A method for establishing both an optical connection and an electrical connection through the drape 201 includes establishing a sterile field with the drape 201; piercing the drape 201 from a sterile side thereof with the electrical terminal or piercing element 266 that extends from the plug 260, wherein the piercing element 266 includes the longitudinal cutout 268 between the housing 262 of the plug 260 and a tip of the piercing element 266 in which cutout 268 the optical terminal 264 that extends from the plug 260 is disposed; inserting the plug 260 into the receptacle 570 or 770 on a non-sterile side of the drape 201, wherein the receptacle 570 or 770 includes the optical receiver 572 or 772 and the electrical receiver 574 or 774 within the receptacle housing 272 configured to form the optical connection with the optical terminal 264 and the electrical connection with the piercing element 266, respectively; and establishing both the optical and electrical connections through the drape 201 without compromising sterility of the sterile field.

Piercing the drape 201 with the piercing element 266 simultaneously advances the optical terminal 264 through the drape 201 in accordance with disposition of the optical terminal 264 in the longitudinal cutout 268 of the piercing element 266.

Inserting the plug 260 into the receptacle 570 or 770 includes inserting the piercing element 266 into the electrical-receiver channel 576 or 776. The electrical-receiver channel 576 or 776 is configured to deflect the piercing element 266 away from the longitudinal axis of the plug 260 or toward the longitudinal side of the plug 260. Deflecting the piercing element 266 away from the longitudinal axis of the plug 260 or toward the longitudinal side of the plug 260 exposes the optical terminal 264.

Inserting the plug 260 into the receptacle 570 or 770 includes forming a butt-joint connection between a ferrule-covered portion of the optical terminal 264 and a ferrule-covered portion of the optical fiber 679 at a closed end of the optical receiver 572 or 772.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A method for making a connection system for establishing both an optical connection and an electrical connection through a drape, comprising:
producing a plug comprising a plug housing, an optical terminal, and an electrical terminal, each of the optical terminal and the electrical terminal extending from the plug housing, the electrical terminal including a piercing tip configured to pierce the drape;
constructing a receptacle comprising a receptacle housing, an optical receiver, and an electrical receiver, wherein:
the receptacle housing is configured to receive the plug housing,
the optical receiver is configured to receive the optical terminal to establish the optical connection, and
the electrical receiver is configured to receive the electrical terminal to establish the electrical connection.

2. The method according to claim 1, wherein the electric terminal includes a cutout between the plug housing and the piercing tip, wherein the optical terminal terminates in the cutout, and wherein the electrical terminal is configured to deflect away from the optical terminal to establish the electrical connection and the optical connection.

3. The method according to claim 2, wherein the receptacle further comprises an optical receiver channel and an electrical receiver channel separate from the optical receiver channel.

4. The method according to claim 3, wherein the electrical receiver channel includes an angled bottom surface having a length to match a length of a side of the electrical terminal.

5. The method according to claim 3, wherein the optical receiver channel has a shape corresponding to a shape of the optical terminal, and wherein the electrical receiver channel has a shape corresponding to a shape of the electrical terminal.

6. The method according to claim 1, wherein the optical terminal includes a first optical fiber extending into a first ferrule, wherein the optical receiver includes a second optical fiber disposed in a second ferrule, and wherein the first ferrule and the second ferrule are configured to connect to establish the optical connection in a straight sleeve-type butt-joint connection.

7. The method according to claim 1, wherein the optical terminal includes a first optical fiber extending into a first ferrule, wherein the optical receiver includes a second optical fiber disposed in a second ferrule, and wherein the first ferrule and the second ferrule are configured to connect to establish the optical connection in a tapered sleeve-type butt-joint connection.
